(19) United States
(12) Patent Application Publication
(10) Pub. No.: US 2014/0018648 A1
(43) Pub. Date: Jan. 16, 2014

(54) BLOOD PARAMETER MEASURING DEVICE AND METHOD FOR MEASURING BLOOD PARAMETER

(71) Applicant: Industrial Technology Research Institute, Hsinchu (TW)

(72) Inventors: Sun-Hua Pao, Taipei City (TW); Chieh-Neng Young, Tainan City (TW); Yio-Wha Shau, Taipei City (TW); Hung-Sen Tsao, Hsinchu City (TW)

(21) Appl. No.: 13/896,347

(22) Filed: May 17, 2013

Related U.S. Application Data
(60) Provisional application No. 61/648,629, filed on May 18, 2012.

(30) Foreign Application Priority Data
Dec. 28, 2012 (TW) 101151066

(51) Int. Cl. A61B 5/1455

(52) U.S. Cl. CPC A61B 5/14552
USPC 600/322; 600/309; 600/364

(57) ABSTRACT

A blood parameter measuring device and a method for measuring a blood parameter are provided. The blood parameter measuring device includes an emitted source, a receiver module, and an actuator. The emitted source is disposed at a side of a tissue to be analyzed and provides at least two different wavelengths of radiation. The receiver module is disposed at another side of the tissue to be analyzed to receive the attenuated radiation produced by the emitted source. The actuator is connected to at least one of the emitted source and the receiver module. The actuator generates a driving force to make the emitted source and the receiver module contacts the tissue to be analyzed, thereby imposing a normal stress on a surface of the tissue to be analyzed to change a wave path between the emitted source and the receiver module.

### CROSS-REFERENCE TO RELATED APPLICATION

This application claims the priority benefits of U.S. provisional application Ser. No. 61/648,629, filed on May 18, 2012 and Taiwan application serial no. 101151066, filed on Dec. 28, 2012. The entirety of each of the above-mentioned patent applications is hereby incorporated by reference herein and made a part of this specification.

### BACKGROUND

1. Technical Field

The technical field relates to a blood parameter measuring device and a method for measuring a blood parameter.

2. Description of Related Art

Various types of systems have been developed for analyzing the concentrations of blood constituents, such as blood glucose, blood oxygen, medicine, carboxyhemoglobin, methemoglobin, and cholesterol, etc., which play an important role in terms of health assessment or detection of special diseases.

Generally speaking, the measurement of a blood parameter requires the process of blood drawing for analyzing the blood, and the parameter analysis is done outside the human body. However, blood drawing may be inapplicable in some cases (for people having allergy or anemia, for example). Therefore, a non-invasive blood parameter measuring technique is required.

Take blood oxygen meter as an example, a typical non-invasive blood oxygen meter calculates the oxyhemoglobin saturation by pulse oximetry (SpO₂) using an optical detection method based on the variation of the blood volume in the analyzed part due to pulse, so as to measure the concentration of blood oxygen. Nevertheless, in the case that tissue perfusion due to pulse does not occur, it may be difficult to accurately measure SpO₂ even though theoretically oxygenated hemoglobin (HbO₂) still exists in the tissue. To meet the actual application, it is required to further improve the non-invasive blood parameter detection technique.

### SUMMARY

The disclosure provides a blood parameter measuring device, which changes a wave path of a wave that passes through a tissue to be analyzed in an active way for measuring a blood parameter.

The disclosure provides a blood parameter measuring method for measuring a blood parameter by actively changing a wave path of a wave that passes through a tissue to be analyzed.

The disclosure provides a blood parameter measuring device adapted for measuring a blood parameter of a tissue to be analyzed, and the blood parameter measuring device includes: an emitted source disposed at a side of the tissue to be analyzed and providing at least two different wavelengths of radiation; a receiver module disposed at another side of the tissue to be analyzed to receive the radiation generated by the emitted source, wherein the radiation is attenuated; and an actuator connected to at least one of the emitted source and the receiver module. The actuator generates a normal stress to change a wave path between the emitted source and the receiver module. Because the tissue to be analyzed is compressed back and forth by the normal stress, the blood volume in the tissue to be analyzed is varied due to the compression and backflow of the blood, thereby changing a wave path between the emitted source and the receiver module.

According to an embodiment of the disclosure, in the blood parameter measuring device, the normal stress is between a diastolic blood pressure and a systolic blood pressure of the tissue to be analyzed.

According to an embodiment of the disclosure, in the blood parameter measuring device, the normal stress is caused by a mechanical force, an electromagnetic force, or a combination of the foregoing.

According to an embodiment of the disclosure, the blood parameter measuring device further includes an operation module that is at least coupled to the receiver module to analyze signals of the attenuated radiation received by the receiver module, wherein a linear combination of the two different wavelengths of the attenuated radiation is designed as a threshold to distinguish signals came from pure blood perfusion or other disturbances.

According to an embodiment of the disclosure, in the blood parameter measuring device, the operation module includes a feedback control unit, a data calculation unit, a data transmission unit, and a data display unit.

According to an embodiment of the disclosure, the blood parameter measuring device further includes a pressure sensor configured for measuring the normal stress generated on the tissue to be analyzed.

According to an embodiment of the disclosure, the blood parameter measuring device further includes a support mechanism that is a movable mechanism and connected to the actuator, and at least one of the emitted source and the receiver module is disposed on the support mechanism.

According to an embodiment of the disclosure, in the blood parameter measuring device, the support mechanism has a clip type, circularly wrapped, or planar attached structure.

According to an embodiment of the disclosure, in the blood parameter measuring device, the actuator generates the normal stress according to a time function.

According to an embodiment of the disclosure, in the blood parameter measuring device, the blood parameter includes a blood oxygen concentration.

The disclosure provides a blood parameter measuring method for measuring a blood parameter of a tissue to be analyzed, and the blood parameter measuring method includes: emitting at least two radiated waves from an emitted source to pass through the tissue to be analyzed, wherein the radiated waves have different wavelengths; detecting the attenuated signals of the radiation waves from the tissue to be analyzed through a receiver module and continuously generating output signals; generating a normal stress through an actuator to change relative wave path between the emitted source and the receiver module to affect the output signals; and analyzing the output signals to obtain the blood parameter of the tissue to be analyzed.

According to an embodiment of the disclosure, in the blood parameter measuring method, the radiated wave includes an electromagnetic wave, a mechanical wave, or a combination of the foregoing.

According to an embodiment of the disclosure, in the blood parameter measuring method, the normal stress is caused by a mechanical force, an electromagnetic force, or a combination of the foregoing.

According to an embodiment of the disclosure, in the blood parameter measuring method, at least one of the emitted source and the receiver module is disposed on a support mechanism, which is a movable mechanism and connected to the actuator.

According to an embodiment of the disclosure, in the blood parameter measuring method, the actuator generates the normal stress according to a time function.

According to an embodiment of the disclosure, in the blood parameter measuring method, the normal stress is applied to compress the tissue with a pressure higher than a local diastolic blood pressure, and is continuously increased to reach a local systolic blood pressure at a first phase, then is released naturally at a second phase.

According to an embodiment of the disclosure, in the blood parameter measuring method, the output signals are normalized at the first phase, and the difference between the output signals are analyzed for blood parameters at the second phase.

Based on the above, the blood parameter measuring device and method of the disclosure measure the blood parameter by actively changing the wave path of the waves that pass through the tissue to be analyzed. Thus, in addition to the case that the tissue has tissue perfusion caused by pulse, the blood parameter measuring device and method are also applicable to a tissue that has no pulse or has feeble pulse.

To make the aforementioned and other features and advantages of the disclosure more comprehensible, several embodiments accompanied with figures are described in detail below.

### BRIEF DESCRIPTION OF THE DRAWINGS

The accompanying drawings are included to provide a further understanding of the disclosure, and are incorporated in and constitute a part of this specification. The drawings illustrate exemplary embodiments and, together with the description, serve to explain the principles of the disclosure.

FIG. 1 is a schematic view of a blood parameter measuring device according to an embodiment of the disclosure.

FIG. 2 is a schematic view of a blood parameter measuring device according to an embodiment of the disclosure.

FIG. 3 is a flowchart showing a method for measuring a blood parameter according to an embodiment of the disclosure.

FIG. 4 illustrates an infrared light signal/red light signal obtained using a blood parameter measuring device according to an embodiment of the disclosure in the case of normal tissue perfusion.

FIG. 5 illustrates an infrared light signal/red light signal obtained when a blood parameter measuring device according to an embodiment of the disclosure is applied on a fingertip having no tissue perfusion (a pressure cuff is used on the upper arm to block the perfusion to the fingertip).

FIG. 6 illustrates an infrared light signal/red light signal obtained when a blood parameter measuring device according to an embodiment of the disclosure is applied on a fingertip having no tissue perfusion (a pressure cuff is used on the upper arm to block the perfusion to the fingertip). The infrared and red light signals are normalized in a first interval. Then, during a second interval in which natural backflow of the blood occurs in a tissue to be analyzed, a difference D(Red-IR) is generated, which is resulted from the difference in absorbance of the red light (with a wavelength of 660 nm) and the infrared light (with a wavelength of 940 nm) by blood oxygen.

FIG. 7 illustrates a blood parameter measuring device according to an embodiment of the disclosure which is applied on a fingertip having no tissue perfusion. It is a partial enlarged view in the second interval wherein three light sources (with wavelengths of 660 nm, 805 nm, and 940 nm) are adopted.

FIG. 8 illustrates a partial enlarged view of FIG. 7, in which a blood parameter measuring device according to an embodiment of the disclosure is applied on a fingertip having no tissue perfusion. A ratio of the difference in absorbance (D(Red)/D(IR)) of three light sources by blood oxygen is measured.

FIGS. 9(a) to 9(c) illustrate a blood parameter measuring device according to an embodiment of the disclosure which is applied on a fingertip having no tissue perfusion. If signals are disturbed by external disturbance, as a mechanism for eliminating disturbed signals, an error factor can be calculated after performing a linear combination of red light and infrared light, thereby ensuring the reliability of calculation results of the blood parameter.

### DETAILED DESCRIPTION OF DISCLOSED EMBODIMENTS

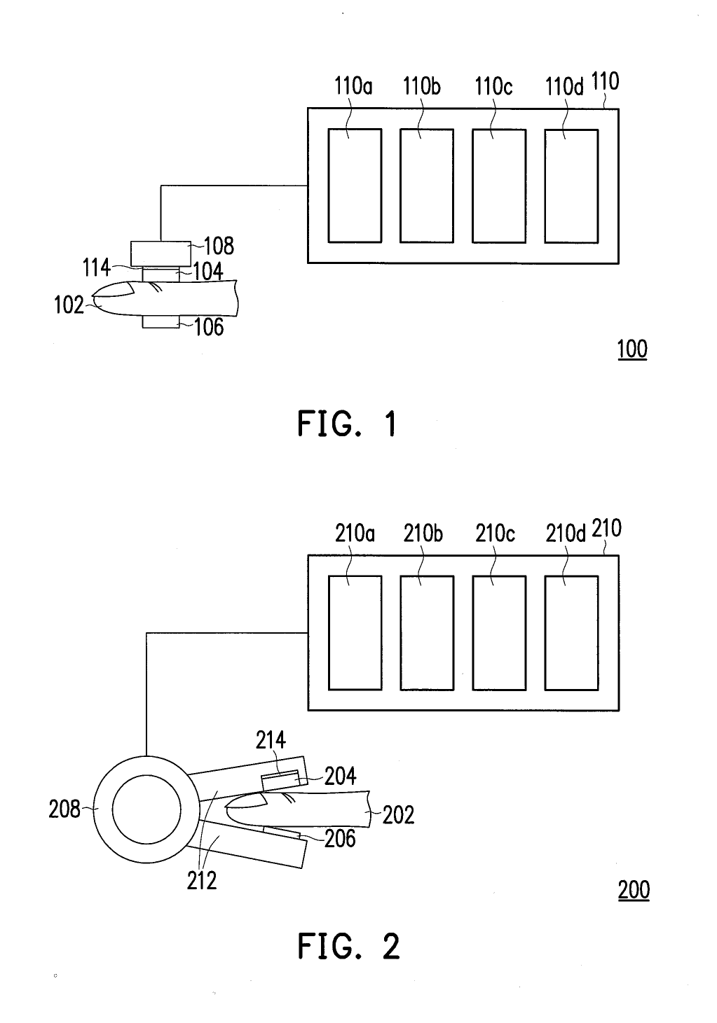

FIG. 1 is a schematic view of a blood parameter measuring device according to an embodiment of the disclosure.

Referring to FIG. 1, a blood parameter measuring device 100 is adapted for measuring a blood parameter of a tissue 102 that is to be analyzed. The tissue 102 that is to be analyzed may be a finger as shown in FIG. 1, for example, but is not limited thereto. In other embodiments, the tissue 102 may be a toe, ear (earlobe), tongue, or any other parts that contain blood, which may change according to the actual requirement. In addition, the blood parameter mentioned in the disclosure may include the content or concentration of blood glucose, blood oxygen, medicine, carboxyhemoglobin, methemoglobin, and cholesterol, etc., but is not limited to the foregoing.

As shown in FIG. 1, the blood parameter measuring device 100 includes an emitted source 104, a receiver module 106, and an actuator 108. The emitted source 104 is disposed at a side of the tissue 102 that is to be analyzed and provides at least two waves which have different wavelengths. The waves may be electromagnetic waves, mechanical waves, or a combination of the foregoing, for example.

The emitted source 104 is able to provide at least two different wavelengths of radiation. For example, the emitted source 104 provides a red light of 660 nm or an infrared light of 940 nm, but is not limited thereto. In some embodiments, the emitted source 104 at least provides the infrared light and the red light. For instance, when assessing the blood oxygen concentration of the tissue to be analyzed, a ratio of oxygenated hemoglobin to deoxygenated hemoglobin is calculated by analyzing the light intensities of the infrared light (having wavelength of 700 nm-14,00 nm) and the red light (having wavelength of 600 nm-700 nm) that pass through the tissue.

The receiver module 106 is disposed at another side of the tissue 102 to be analyzed to receive the waves (or the radiation) produced by the emitted source 104. It is known that a wave is easily attenuated as it propagates through a medium; therefore, these waves (or radiation) received by the receiver module 106 are attenuated waves (or radiation) as they already propagate through the tissue to be analyzed before reaching the receiver module 106. The receiver module 106 is a luminosity sensor, for example, but not limited thereto.

The actuator 108 is connected to at least one of the emitted source 104 and the receiver module 106. In this embodiment, the actuator 108 is connected to the emitted source 104, for instance. The actuator 108 is, for example, a mechanism which is repetitively driven by a motor to press the tissue, but not limited thereto.

The actuator 108 generates a driving force to make the emitted source 104 and the receiver module 106 contact the tissue 102 to be analyzed, thereby imposing a normal stress on a surface of the tissue 102 to be analyzed, and changing a wave path between the emitted source 104 and the receiver module 106.

The driving force is a mechanical force, an electromagnetic force, or a combination of the foregoing, for example. More specifically, the driving force includes an elastic force, an air pressure force, a liquid pressure force, an inertial force, an electromagnetic force, or a combination of the foregoing. In practice, the normal stress caused by the driving force may be a stress that is sufficient to change the wave path between the emitted source 104 and the receiver module 106, but is not limited to the above.

Moreover, the actuator 108 may generate the normal stress according to a time function. The time function is a function having periodicity, regularity, or specific time, for example. More specifically, a periodic square wave may serve as the time function to set the actuator for generating the normal stress required.

It should be noted that the normal stress is higher than a diastolic blood pressure of the tissue 102 to be analyzed, for example. The accuracy of the measurement is further improved when the normal stress is maintained in this range. In addition, because it is not required to impose an excessive pressure on a large area of the tissue 102, the subject may feel more comfortable during measurement.

As shown in FIG. 1, the blood parameter measuring device 100 may further include an operation module 110, which is at least coupled to the receiver module 106 for analyzing a signal received by the receiver module 106. The operation module 110 is a computer host system, for example, but not limited thereto. In some embodiments, the operation module 110 includes a feedback control unit 110a, a data calculation unit 110b, a data transmission unit 110c, and a data display unit 110d.

In the operation module 110 according to an embodiment of the disclosure, the data calculation unit 110b may perform operations, such as performing calculation on a wave signal received by the receiver module 106 according to an algorithm that is set according to the requirement. Then, a calculation result is transmitted to the data display unit 110d through the data transmission unit 110c, so as to obtain a required measurement value. The feedback control unit 110a may obtain the calculation result through at least one of the data calculation unit 110b, the data transmission unit 110c, and the data display unit 110d and determines whether the calculation result has certain stability or reliability.

In some embodiments, the blood parameter measuring device 100 may further include a pressure sensor 114 configured for measuring the normal stress generated on the tissue to be analyzed. The pressure sensor 114 is a piezoelectric material, for example, but not limited thereto.

In this embodiment, the pressure sensor 114 is disposed on the emitted source 104 and senses the normal stress transmitted through the emitted source 104. However, the position of the pressure sensor 114 is not limited to the above, and the pressure sensor 114 may be disposed in other positions (on the receiver module 106, for example) as long as the pressure sensor 114 can sense and measure the normal stress.

More specifically, the pressure sensor 114, for example, transmits a measured pressure value to the feedback control unit 110a of the operation module 110 for the feedback control unit 110a to determine the stability or reliability of the obtained measurement value.

If the obtained measurement value is not stable or reliable enough, the feedback control unit 110a is able to transmit a signal to the actuator 108 and enable the actuator 108 to apply a pressure on the tissue to be analyzed again for measurement. Otherwise, the feedback control unit 110a may send a notification signal to the data display unit 110d to remind the operator to adjust conditions such as environment parameters (e.g. the algorithm, time, and normal stress), so as to facilitate the measurement.

Moreover, the aforementioned algorithm set according to the requirement is not limited to the above disclosure and may be set according to the blood parameter that needs to be measured. For example, when assessing the blood oxygen concentration of the tissue that is to be analyzed, because HbO₂ in the blood absorbs more infrared light and less red light, and Hb absorbs more red light and less infrared light, a peak-valley method, i.e. the Beer-Lambert Law, may be used to detect a variation of light absorption of the blood, so as to calculate a percentage of HbO₂ in the total hemoglobin, thereby obtaining SpO₂.

As described above, the blood parameter measuring device of the disclosure measures the blood parameter by actively changing the wave path of the waves that pass through the tissue to be analyzed. Thus, in addition to the case that the tissue has tissue perfusion caused by pulse, the blood parameter measuring device is also applicable to a tissue that has no pulse or has feeble pulse.

FIG. 2 is a schematic view of a blood parameter measuring device according to an embodiment of the disclosure. In FIG. 2, components similar to those of FIG. 1 are denoted by similar reference numerals (e.g. emitted source 104 and emitted source 204), and detailed descriptions thereof are omitted hereinafter.

Referring to FIG. 2, in this embodiment, a blood parameter measuring device 200 includes an emitted source 204, a receiver module 206, an actuator 208, a support mechanism 212, and a pressure sensor 214. In this embodiment, the actuator 208 is a disc actuator.

A main difference between the blood parameter measuring device 200 and the blood parameter measuring device 100 lies in that: the blood parameter measuring device 200 further includes the support mechanism 212, which is a movable mechanism and connected to the actuator 208. In this embodiment, the support mechanism 212 has a clip type structure, but the disclosure is not limited thereto. The support mechanism 212 may also have a circularly wrapped or planar attached structure.

In this embodiment, the emitted source 204 and the receiver module 206 are both disposed on the support mechanism 212. Accordingly, the actuator 208 may move the support mechanism 212 to drive the emitted source 204 and the receiver module 206, so as to change relative positions thereof. However, the configuration of the emitted source, the receiver module, and the support mechanism is not limited to the above and may be altered as long as at least one of the emitted source and the receiver module is disposed on the support mechanism.

In addition, other technical content, material, and characteristics of the blood parameter measuring device of this embodiment have been specified in the previous embodiment. Hence, a detailed description thereof is omitted hereinafter.

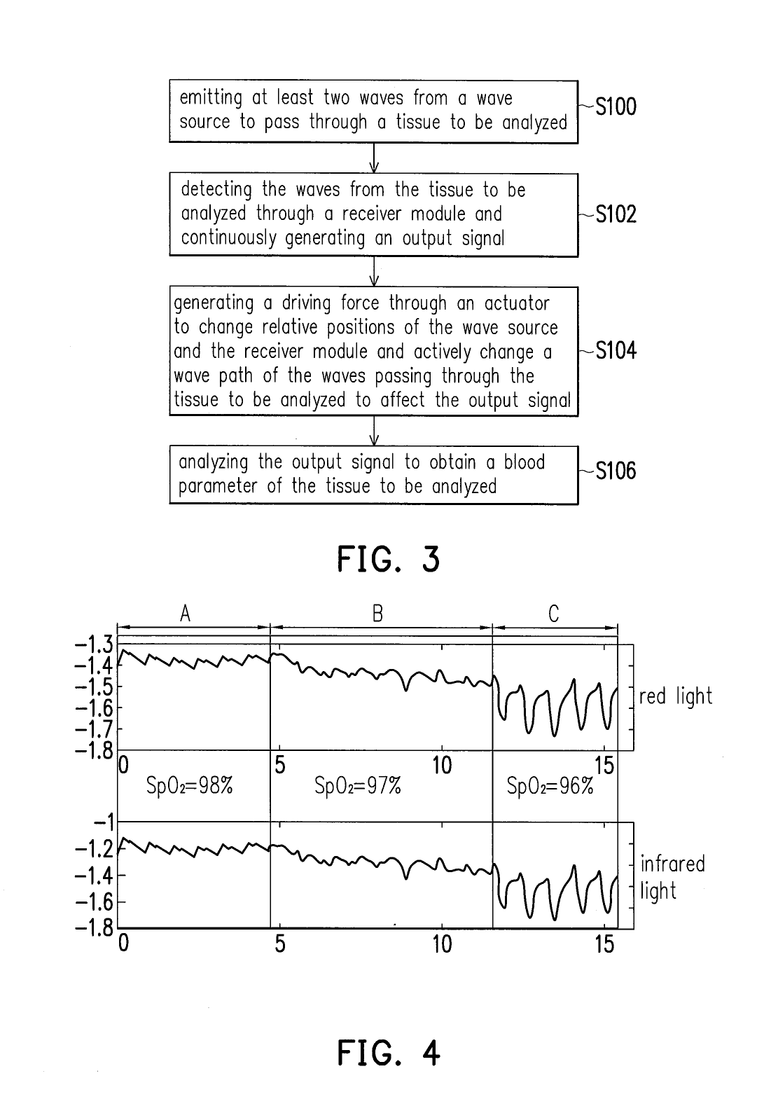

FIG. 3 is a flowchart showing a method for measuring a blood parameter according to an embodiment of the disclosure. With reference to the blood parameter measuring device of FIG. 1, a method for measuring a blood parameter according to an embodiment of the disclosure is described below based on FIG. 3. It is noted that, since some components have been specified in the previous embodiments, descriptions thereof will be omitted hereinafter.

Referring to FIG. 1 and FIG. 3, first, Step S100 is performed, in which the emitted source 104 emits at least two waves to pass through the tissue 102 that is to be analyzed, and the waves have different wavelengths. The waves may be electromagnetic waves, mechanical waves, or a combination of the foregoing, for example. In some embodiments, the waves emitted by the emitted source 104 at least include an infrared light and a red light, for assessing the blood oxygen concentration of the tissue to be analyzed, but the disclosure is not limited thereto. In actual application, when other blood parameters of the tissue are assessed, waves having a different wavelength or a combination of waves of different wavelengths may be used to measure specific blood parameters.

Next, Step S102 is carried out, in which the receiver module 106 detects the waves from the tissue 102 to be analyzed and continuously generates output signals. The output signals are, for example, processed and transmitted by the operation module 110 shown in FIG. 1 and are continuously displayed by the data display unit 110d, but the disclosure is not limited thereto. The output signals may also be processed and presented in a way that is known to those skilled in the art.

Then, Step S104 is performed, in which the normal stress caused by the driving force, that is generated by the actuator 108, changes the relative positions of the emitted source 104 and the receiver module 106, so as to actively change the wave path of the waves that pass through the tissue to be analyzed and affect the output signals. The driving force is a mechanical force, an electromagnetic force, or a combination of the foregoing, for example. The actuator generates the normal stress, for example, according to a time function. Details thereof have been specified in the previous embodiments and thus will be omitted hereinafter.

In some embodiments, at least one of the emitted source 104 and the receiver module 106 is disposed on the support mechanism, which is a movable mechanism and connected to the actuator 108. The actuator 108 may move the support mechanism to drive the emitted source 104 and the receiver module 106, so as to change the relative distance of the emitted source 104 and the receiver module 106.

In addition, the change of the relative distance of the emitted source 104 and the receiver module 106, for example, includes making the emitted source 104 and the receiver module 106 contact the tissue 102 that is to be analyzed, thereby imposing the normal stress on the surface of the tissue 102 to be analyzed during a first phase, and stopping applying the stress to the tissue 102 to be analyzed during the second phase. More specifically, the stress on the tissue 102 to be analyzed is released, for example, after the first phase that the tissue 102 is pressed to reduce the blood volume therein. During the second phase that application of the stress is stopped, blood flows back to the tissue 102, and the output signals obtained at this moment is more suitable for the analysis of the blood parameter. For assessing the blood oxygen concentration, the output signals may be analyzed in a second time section before the second phase becomes stable (a phase when blood flows back), and based on the difference in absorbance of multiple light sources, a final calculation result can be obtained.

It is noted that, because the wave path of the waves that pass through the tissue to be analyzed is actively changed, the measuring method is also applicable to a tissue that has no pulse or has feeble pulse besides the case that the tissue has tissue perfusion caused by pulse, and thus the measuring method is preferred.

As described above, the normal stress is higher than the diastolic blood pressure of the tissue 102 to be analyzed, for example. The accuracy of the measurement is further improved when the normal stress is maintained within this range. In addition, because it is not required to impose an excessive pressure on a large area of the tissue 102, the subject may feel more comfortable during measurement. Furthermore, a method for applying the pressure, for example, includes continuously increasing pressure on the tissue 102 to be analyzed until the imposed pressure reaches a systolic blood pressure of the tissue 102.

Thereafter, Step S106 is performed to analyze the output signals so as to obtain the blood parameter of the tissue 102 to be analyzed. Specifically, the output signals are analyzed by the operation module 110 shown in FIG. 1. A method of analyzing the output signals may have different settings according to the requirement of measurement. For instance, in order to assess the blood oxygen concentration, the output signals may be analyzed in a second time section before the second phase becomes stable (a phase when blood flows back), and a final calculation result can be obtained based on the difference in absorbance of multiple light sources. To be more specific, analyzing the output signals may include performing a linear combination of the two different wavelengths of the attenuated radiation, and the result of the linear combination can be designed as a threshold to distinguish signals came from pure blood perfusion or other disturbances.

It should be noted that, after the output signals are analyzed, the measuring method may further include a step of determining whether the obtained analysis result shows certain stability or reliability. This step is performed by the feedback control unit 110a of FIG. 1, for example. As mentioned above, if the obtained measurement value is not stable or reliable enough, the feedback control unit 110a can transmit a signal to the actuator 108 and enable the actuator 108 to apply a pressure on the tissue to be analyzed again for measurement. Otherwise, the feedback control unit 110a may send a notification signal to the data display unit 110d to remind the operator to adjust conditions such as environment parameters (e.g. the algorithm, time, and normal stress), so as to facilitate the measurement. By this mechanism, the measurement result is optimized.

Moreover, the measuring method of the disclosure may further include measuring the normal stress generated on the tissue 102 to be analyzed through a pressure sensor, for adjusting the conditions described above. Accordingly, whether the pressure applied on the tissue 102 to be analyzed is within an optimal range can be confirmed for obtaining an optimal measurement result.

As described above, the blood parameter measuring method of the disclosure measures the blood parameter by actively changing the wave path of the waves that pass through the tissue to be analyzed. Thus, in addition to the case that the tissue has tissue perfusion caused by pulse, the blood parameter measuring method is also applicable to a tissue that has no pulse or has feeble pulse.

In the following paragraphs, results of experiments are provided as examples for further explaining the disclosure. In the experiments, the blood oxygen concentration of the tissue to be analyzed was assessed by calculating the SpO₂ value, for example. It should be noted that the following experiments show results that were obtained by the blood parameter measuring device of the disclosure under specific conditions and thus should not be construed as limitations to the scope of the disclosure.

Experiment 1

FIG. 4 illustrates an infrared light signal/red light signal obtained using the blood parameter measuring device according to an embodiment of the disclosure in the case of normal tissue perfusion.

With reference to FIG. 4, in Region A on the left (about the 0^{th} to the 5^{th} second), the tissue to be analyzed was in a state of normal perfusion (that is, the tissue had normal pulse and was applied with no pressure). Because the blood pressure and blood stream naturally changed the optical path difference of red light/infrared light (Red/IR), the receiver module received output signals with continuous and regular pulse to calculate the SpO₂ value. The SpO₂ value measured in Region A was about 98%.

Starting from the 5^{th} second, the driving force generated by the actuator changed the relative positions of the emitted source and the receiver module and imposed the normal stress on the tissue to be analyzed to actively change the optical path difference and affect the output signals. The normal stress applied here was approximately equal to the systolic blood pressure, so as to obtain the largest reduction in blood volume. Thus, in Region B in the middle (about the 5^{th} to the 12^{th} second), the influence brought by the active change of the optical path difference was mixed with the pulse of the normal blood pressure and blood stream. Although the original regularity was changed, the SpO₂ value could still be obtained based on the output signals. The SpO₂ value measured in Region B was about 97%.

Starting from the 12^{th} second, a normal stress (about 150 mmHg in this experiment, which is much greater than the systolic blood pressure) greater than the normal stress in Region B was applied on the surface of the tissue to be analyzed. The result showed that, in Region C on the right, because the influence that the active change of the optical path difference caused to the output signals was much greater than the influence caused by the pulse of the normal blood pressure and blood stream, the result of the output signals were dominated by the actively changed optical path difference and a waveform different from the waveform of Region B was formed. This waveform could also be used to calculate the SpO₂ value. The SpO₂ value measured in Region C was about 96%. It should be noted that, in this case, the distance between the peak and the valley became more obvious, which was more suitable for the analysis of blood oxygen concentration.

Experiment 2

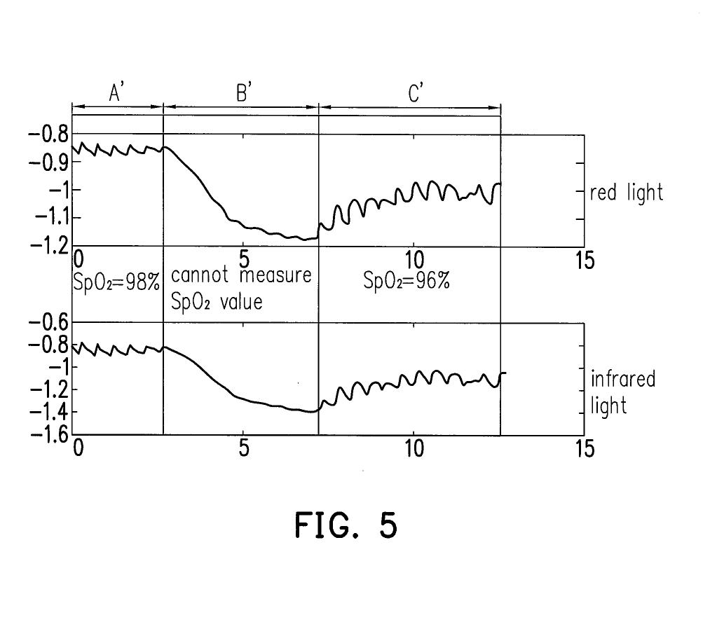

FIG. 5 illustrates an infrared light signal/red light signal obtained when the blood parameter measuring device according to an embodiment of the disclosure is applied on a fingertip having no tissue perfusion (a pressure cuff is used on the upper arm to block the perfusion to the fingertip).

With reference to FIG. 5, in Region A′ on the left (about the 0^{th} to the 2^{nd} second), the tissue to be analyzed was in the state of normal pulse. The Region A′ was the same as Region A in the Experiment 1 that the receiver module could receive the output signals with continuous and regular pulse to calculate the SpO₂ value. The SpO₂ value measured in Region A′ was about 98%.

Starting from the 2^{nd} second, a cuff was used on the upper arm to increase the applied pressure to the systolic blood pressure (the pressure applied here was approximately equal to the systolic blood pressure, such that the pulse could not be transmitted to the fingertip that is to be analyzed). That is to say, in Region B′, the tissue gradually entered a state of no perfusion. Therefore, in Region B′ in the middle, decrease of the signal could be observed. Since the actuator was not used to change the relative positions of the emitted source and the receiver module, the surface of the tissue to be analyzed was not compressed by the normal stress, and the tissue remained in the state of no perfusion. As a result, in Region B′, the SpO₂ value could not be measured.

Starting from the 7^{th} second, the driving force generated by the actuator changed the relative positions of the emitted source and the receiver module to impose the normal stress on the surface of the tissue to be analyzed and actively change the optical path difference to affect the output signals. The normal stress applied here was approximately equal to the systolic blood pressure. Therefore, in Region C′ on the right, because the actuator was used to actively move the position of the emitted source and cause the signal received by the receiver module to change, the output signals having continuous and regular pulse was generated again. Accordingly, the SpO₂ value could be calculated for assessing blood oxygen concentration. The SpO₂ value measured in Region C′ was about 96%.

Experiment 3

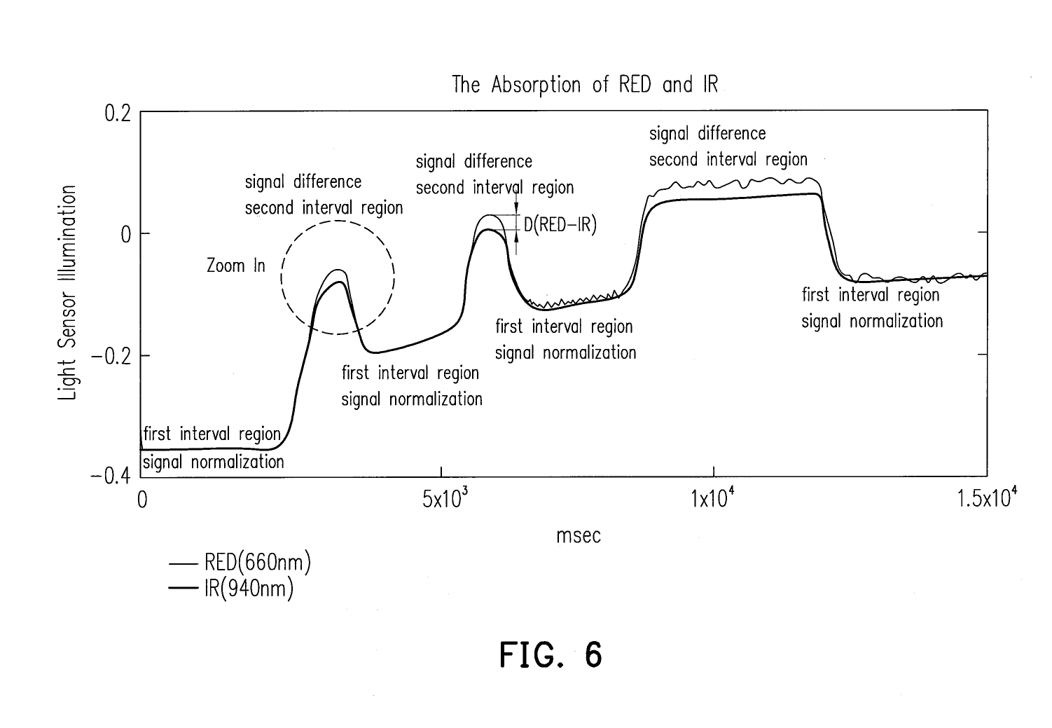

FIG. 6 illustrates an infrared light signal/red light signal obtained when a blood parameter measuring device according to an embodiment of the disclosure is applied on a fingertip having no tissue perfusion (a pressure cuff is used on the upper arm to block the perfusion to the fingertip). At first phase, a normal stress was applied on the tissue to be analyzed, and intensity signals of the red light and infrared light were normalized. Since blood oxygen in the tissue had different absorbance of the red light and the infrared light, in the second time section before the second phase became stable (a phase when blood flowed back), light paths of the red light and the infrared light were different. The optical path difference D(Red-IR) has a linear relationship with the oxygen content in blood; therefore, a table lookup method can be established according to experimental results, and the blood parameter may be estimated based on the value of the optical path difference D(Red-IR).

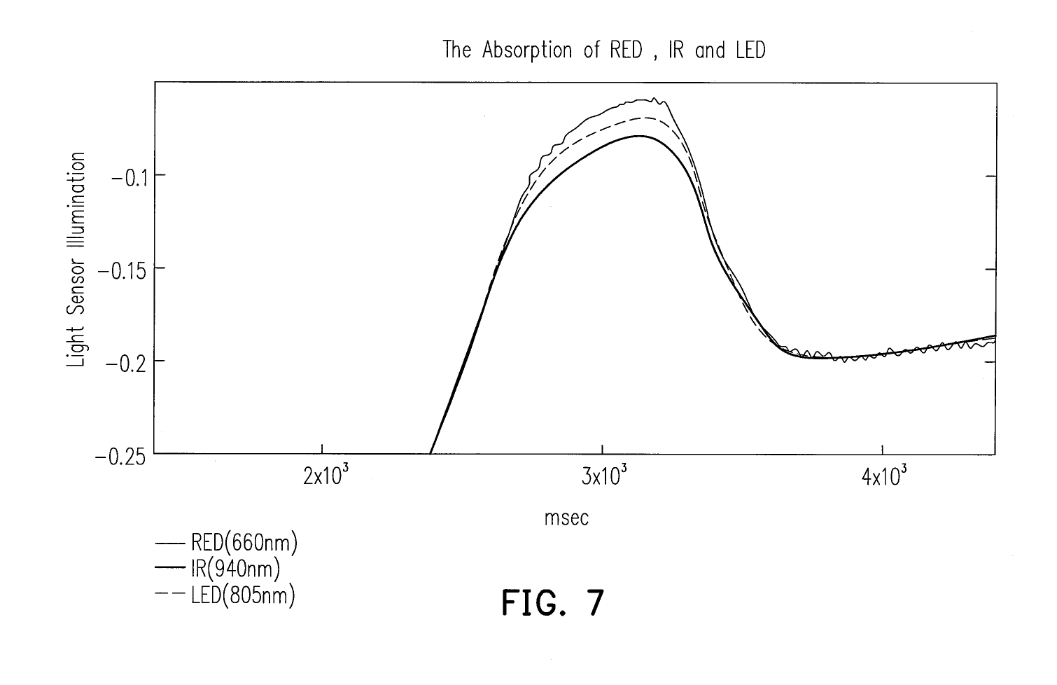

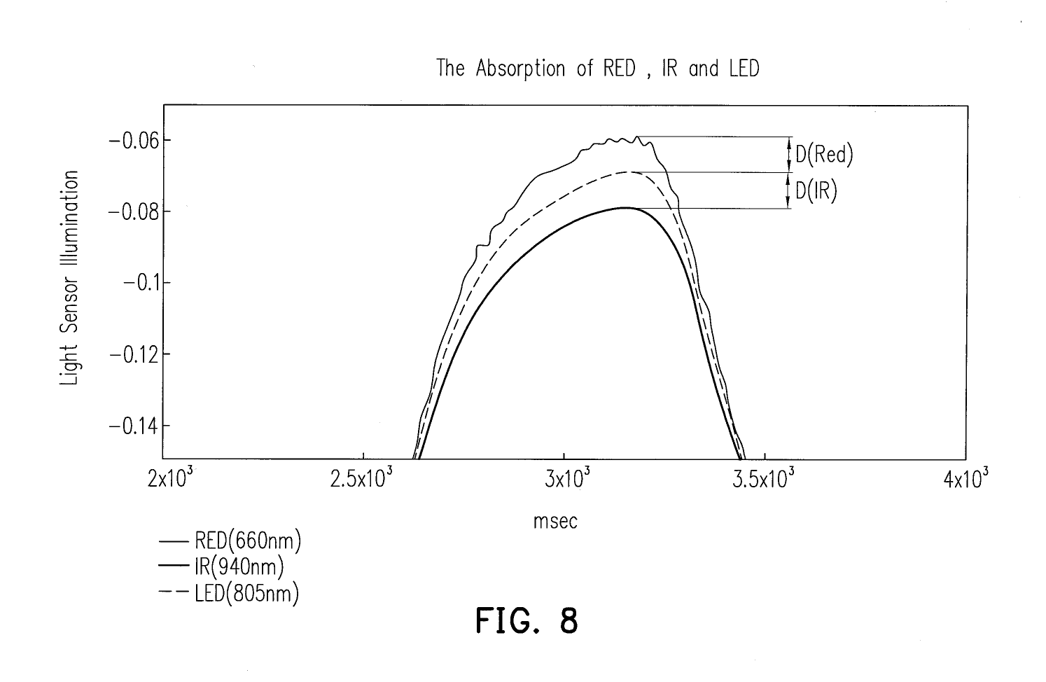

As shown in FIG. 7, it is certainly that using two or more light sources may realize a more specific analysis. As shown in FIG. 8, the optical path difference D(Red) between light with a wavelength of 660 nm and light with a wavelength of 805 nm, and the optical path difference D(IR) between light with a wavelength of 905 nm and light with a wavelength of 805 nm were calculated. The blood parameters can be estimated more precisely based on analysis of the D(Red)/D(IR) ratio. Furthermore, using more light sources may directly reduce error range (caused by tissue heterogeneity) in calculation.

Experiment 4

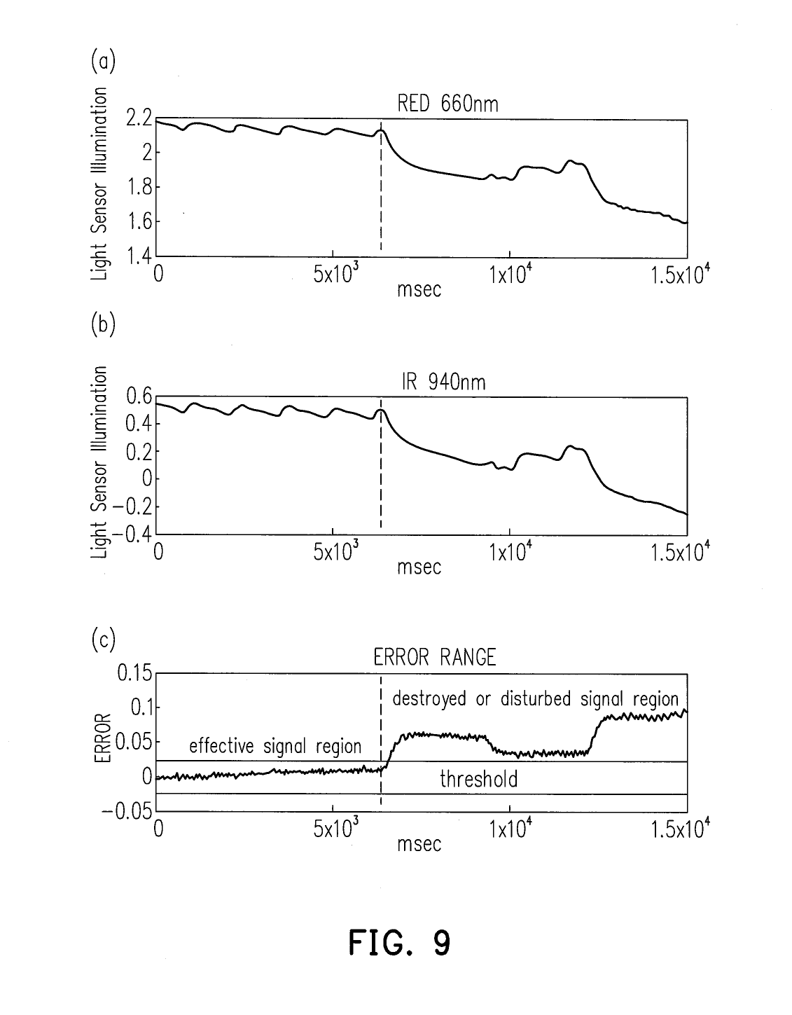

In the embodiment, a blood parameter measuring device according to an embodiment of the disclosure was applied on a fingertip having no tissue perfusion. In some cases, red light and infrared light are easily interfered by an actuator or other external disturbances, thereby obtaining a wrong value of blood parameters; therefore, a method for eliminating error signals is further provided herein. Linear combination of two or more light sources were performed, for example, a formula of E(t)=Red(t)+[K1+K2*IR(t)] could be used. In above formula, E(t) represents an error standard (as shown in FIG. 9(c)), Red(t) represents a red light signal (as shown in FIG. 9(a)), IR(t) represents a infrared light signal (as shown in FIG. 9(b)), and K1 and K2 represent adjusting parameters for adjusting translational motion and ratio of the infrared light, respectively. Since a proper ratio of the red light and infrared light comes from the difference in absorbance by blood oxygen, it is designed to distinguish the changing of signals came from pure blood perfusion or other disturbances. As shown in FIG. 9(c), if E(t) is under a given threshold value, the measured signal can be guaranteed to be correct without any disturbance.

To conclude the above, the blood parameter measuring method of the disclosure measures the blood parameter by actively changing the wave path of the waves that pass through the tissue to be analyzed. Thus, in addition to the case that the tissue has tissue perfusion caused by pulse, the blood parameter measuring method is also applicable to a tissue that has no pulse or has feeble pulse.

It will be apparent to those skilled in the art that various modifications and variations can be made to the disclosed embodiments without departing from the scope or spirit of the disclosure. In view of the foregoing, it is intended that the disclosure covers modifications and variations of this disclosure provided that they fall within the scope of the following claims and their equivalents.

What is claimed is:

1. A blood parameter measuring device, adapted for measuring a blood parameter of a tissue to be analyzed, the blood parameter measuring device comprising:
an emitted source disposed at a side of the tissue to be analyzed and providing at least two different wavelengths of radiation;
a receiver module disposed at another side of the tissue to be analyzed and receiving the radiation generated by the emitted source, wherein the radiation is attenuated; and
an actuator connected to at least one of the emitted source and the receiver module and generating a normal stress to change a wave path between the emitted source and the receiver module.

2. The blood parameter measuring device according to claim 1, wherein the normal stress is between a diastolic blood pressure and a systolic blood pressure of the tissue to be analyzed.

3. The blood parameter measuring device according to claim 1, wherein the normal stress is caused by a mechanical force, an electromagnetic force, or a combination of the foregoing.

4. The blood parameter measuring device according to claim 1, further comprising an operation module that is at least coupled to the receiver module to analyze signals of the attenuated radiation received by the receiver module, wherein a linear combination of the two different wavelengths of the attenuated radiation is designed as a threshold to distinguish signals came from pure blood perfusion or other disturbances.

5. The blood parameter measuring device according to claim 4, wherein the operation module comprises a feedback control unit, a data calculation unit, a data transmission unit, and a data display unit.

6. The blood parameter measuring device according to claim 1, further comprising a pressure sensor configured for measuring the normal stress generated on the tissue to be analyzed.

7. The blood parameter measuring device according to claim 1, further comprising a support mechanism that is a movable mechanism and connected to the actuator, and at least one of the emitted source and the receiver module is disposed on the support mechanism.

8. The blood parameter measuring device according to claim 7, wherein the support mechanism has a clip type, circularly wrapped, or planar attached structure.

9. The blood parameter measuring device according to claim 1, wherein the actuator generates the normal stress according to a time function.

10. The blood parameter measuring device according to claim 1, wherein the blood parameter comprises a blood oxygen concentration.

11. A blood parameter measuring method, adapted for measuring a blood parameter of a tissue to be analyzed, the blood parameter measuring method comprising:
emitting at least two radiated waves from an emitted source to pass through the tissue to be analyzed, wherein the radiated waves have different wavelengths;
detecting attenuated signals of the radiation waves from the tissue to be analyzed through a receiver module and continuously generating output signals;
generating a normal stress through an actuator to change relative wave path between the emitted source and the receiver module to affect the output signals; and
analyzing the output signals to obtain the blood parameter of the tissue to be analyzed.

12. The blood parameter measuring method according to claim 11, wherein the radiated wave comprises an electromagnetic wave, a mechanical wave, or a combination of the foregoing.

13. The blood parameter measuring method according to claim 11, wherein the normal stress is caused by a mechanical force, an electromagnetic force, or a combination of the foregoing.

14. The blood parameter measuring method according to claim 11, wherein at least one of the emitted source and the receiver module is disposed on a support mechanism, which is a movable mechanism and connected to the actuator.

15. The blood parameter measuring method according to claim 11, wherein the actuator generates the normal stress according to a time function.

16. The blood parameter measuring method according to claim 15, wherein the normal stress is applied to compress the tissue with a pressure higher than a local diastolic blood pressure, and is continuously increased to reach a local systolic blood pressure at a first phase, then is released naturally at a second phase.

17. The blood parameter measuring method according to claim 16, wherein the multiple light source output signals are normalized at the first phase and the difference between the output signals are analyzed for blood parameters at the second phase.
